(19) United States
(12) Patent Application Publication
(10) Pub. No.: US 2013/0269045 A1
(43) Pub. Date: Oct. 10, 2013

(54) HB-EGF DEFICIENT TRANSGENIC ANIMAL AND PRODUCTION METHOD THEREOF

(71) Applicants: Nagasaki University, Nagasaki (JP); Shin Nippon Biomedical Laboratories, Ltd., Kagoshima (JP)

(72) Inventor: Hiroshi Ueda, Nagasaki (JP)

(21) Appl. No.: 13/756,247

(22) Filed: Jan. 31, 2013

(30) Foreign Application Priority Data
Mar. 9, 2012 (JP) 2012-052432

(51) Int. Cl. A61K 49/00

(52) U.S. Cl. CPC A61K 49/0008
USPC 800/3; 800/9; 800/22; 800/12

(57) ABSTRACT

A transgenic animal other than human in which neuropsychiatric disorder condition is developed by the deletion of an HB-EGF gene is obtained. The present invention relates to a transgenic animal other than human in which an HB-EGF gene is deficient and neuropsychiatric disorder condition is developed, and a production method thereof, and a method for screening a therapeutic agent for neuropsychiatric disorder. As a transgenic animal in accordance with the present invention, a transgenic animal in which an HB-EGF gene is specifically deficient in the spiny neurons (striatum, and hippocampus) can be obtained by crossbreeding a transgenic animal that contains a genotype of Gng7^{(+/cre)}, and a transgenic animal that contains a genotype of Hb-egf^{(flox/flox)}.

### BACKGROUND

1. Technical Field

The present invention relates to an HB-EGF deficient type transgenic animal. Specifically, the present invention relates to a transgenic animal other than human in which an HB-EGF gene is specifically deficient in the hippocampal neuron region and a condition of neuropsychiatric disorder is developed. The transgenic animal in accordance with the present invention can be a model animal in which a condition of neuropsychiatric disorder is developed.

2. Related Art

Conventionally, study and treatment have been tried in order to clarify the molecular basis of neuropsychiatric disorder such as depression and schizophrenia, however, the effect of the study and treatment has not been sufficiently obtained. As the cause, it is mentioned that the responsible brain area and mechanism of the development of neuropsychiatric disorder have not been elucidated yet, and that an experimental animal model that is appropriate as the subject has not been present.

In the development of psychiatric disorder, involvement of a growth factor that is involved in growth and plasticity of brain and nerve cells has been suggested. It has known that a heparin-binding EGF-like growth factor (HB-EGF) that is one of growth factors is abundantly present in the forebrain and hippocampus regions. It has known that by the deficiency of an HB-EGF present in the forebrain and hippocampus regions, spine immaturity of nerve cell and downregulation of NMDA receptor subunit is generated in a cortex area, and a general schizophrenia-like symptom is exhibited (PLoS One, 2009. 4 (10): p. e7461).

### NON PATENT LITERATURE

Non Patent Literature 1: PLoS One, 2009. 4(10): p. e7461

### SUMMARY

### Problems to be Solved by the Invention

The involvement of the HB-EGF that is expressed largely in a matured brain in a functional role and neuropsychiatric disorder in the hippocampal neuron region has not been elucidated sufficiently. Further, the involvement of the HB-EGF in memory and learning functions in the hippocampal neuron region has not been elucidated sufficiently either.

That is, it has not been obvious that neuropsychiatric disorder is developed by the deficiency of the HB-EGF in the hippocampal neuron region.

In addition, a transgenic animal other than human in which the HB-EGF is specifically deficient in the hippocampal neuron region and a condition of neuropsychiatric disorder is developed cannot be obtained. Therefore, a screening of a therapeutic agent that is effective for neuropsychiatric disorder caused by specific HB-EGF deficiency in the hippocampal neuron region could not be performed by using a model animal.

That is, an object of the present invention is to obtain a transgenic animal other than human in which a condition of neuropsychiatric disorder is developed by specifically deleting the HB-EGF in the hippocampal neuron region.

Further, another object of the present invention is to perform a screening of a therapeutic agent for neuropsychiatric disorder by using a hippocampal neuron region HB-EGF deficient type transgenic animal other than human that develops a condition of neuropsychiatric disorder.

### Means for Solving the Problems

In order to solve the problem of conventional invention described above, the present inventors produced a hippocampal neuron region HB-EGF deficient type transgenic animal and found that the transgenic animal developed a condition of neuropsychiatric disorder. A transgenic animal in accordance with the present invention can be a model animal that develops a condition of neuropsychiatric disorder.

Further, the present inventors found that a hippocampal neuron region HB-EGF deficient type transgenic animal could be used for a screening of a therapeutic agent for neuropsychiatric disorder relating to hippocampal neurons.

The first aspect of the present invention relates to a transgenic animal other than human in which an HB-EGF gene is deficient in the hippocampal neuron region and neuropsychiatric disorder is developed. An example of the transgenic animal other than human is a transgenic animal of a mammal other than human in which an HB-EGF gene is deficient in the hippocampal neuron region. The transgenic animal of the present invention develops neuropsychiatric disorder by specifically deleting an HB-EGF gene in the hippocampal neuron region.

In the present invention, only in the hippocampal neuron region, the HB-EGF gene can be made deficient. In other tissues of the transgenic animal of the present invention, the HB-EGF gene is expressed normally, therefore, fatal impact cannot be provided in the growth of the transgenic animal. Further, in the present invention, it is not necessary to consider the influence of the deficient of an HB-EGF gene in other tissues, or other sites of the brain tissues, therefore, it can be elucidated the function of the HB-EGF gene in the hippocampal neuron region.

In an preferable embodiment of the present invention, an HB-EGF deficient type transgenic animal other than human can be obtained by crossbreeding a transgenic animal that contains a gene promoter being specifically expressed in the hippocampal neuron region and a Cre gene sequence, and a transgenic animal that contains an HB-EGF gene sandwiched in between LoxP sequences.

In a transgenic animal of the present invention, a gene promoter that specifically is expressed in the hippocampal neuron region is arranged at an upstream of the Cre gene, therefore, only in the hippocampal neuron region, the Cre protein is expressed. The Cre protein plays a role in which an HB-EGF gene sequence sandwiched in between LoxP sequences is excised.

In the present invention, the Cre protein is expressed only in the hippocampal neuron region, therefore, in the transgenic animal of the present invention, the HB-EGF gene is deficient only in the hippocampal neuron region.

In a preferable embodiment of the present invention, a gene promoter is a Gng7 promoter, a CamK II promoter, or an Emx1 promoter. Gng7 promoter, a CamK II promoter, and an Emx1 promoter are promoters being specifically expressed in the hippocampal neuron region. A Gng7 promoter is specifically expressed in particular in the spiny neurons (striatum, and hippocampus) of the brain, therefore, a Gng7 promoter is preferably used as a gene promoter.

In the present invention, by arranging a Gng7 promoter, a CamK II promoter, or an Emx1 promoter at an upstream of the Cre gene, the Cre gene can be specifically expressed in the hippocampal neuron region.

In a preferable embodiment of the present invention, neuropsychiatric disorder is any one of depression, schizophrenia, obsessive-compulsive disorder, attention deficit hyperactivity disorder, pervasive developmental disorder including Asperger's syndrome, Alzheimer disease, learning disability, and long-term memory impairment.

Insufficiency of animal models in clinical researches of depression, schizophrenia, obsessive-compulsive disorder, and attention deficit hyperactivity disorder, pervasive developmental disorder including Asperger's syndrome, Alzheimer disease, learning disability, and long-term memory impairment is a huge obstacle in the basic study and the drug development. Therefore, a transgenic animal other than human that develops depression, schizophrenia, obsessive-compulsive disorder, attention deficit hyperactivity disorder, pervasive developmental disorder including Asperger's syndrome, Alzheimer disease, learning disability, and long-term memory impairment can be used for an animal model in clinical researches of depression, schizophrenia, obsessive-compulsive disorder, attention deficit hyperactivity disorder, pervasive developmental disorder including Asperger's syndrome, Alzheimer disease, learning disability, and long-term memory impairment.

In a preferable embodiment of the present invention, a transgenic animal other than human is a mouse.

A mouse has an advantage in growing rapidly, being a small and thus being easy to deal with. Further, a mouse develops a symptom close to that of the neuropsychiatric disorder of human, therefore, the mouse can be a model animal used for a screening of a therapeutic agent.

The second aspect of the present invention relates to a screening method of a therapeutic agent. A screening method in accordance with the present invention is a method for examining an effect of the improvement of the condition of neuropsychiatric disorder by administering a test substance to a transgenic animal other than human in which an HB-EGF gene is deficient. By using a transgenic animal in which an HB-EGF gene is deficient in accordance with the present invention, the screening of a therapeutic agent for neuropsychiatric disorder can be effectively performed.

The third aspect of the present invention relates to a production method of a transgenic animal other than human in which an HB-EGF gene is deficient. A production method in accordance with the present invention is a method for obtaining a transgenic animal in which an HB-EGF gene is deficient by crossbreeding a first transgenic animal other than human that contains a gene promoter being specifically expressed in the hippocampal neuron region and a Cre gene sequence, and a second transgenic animal other than human that contains an HB-EGF gene sandwiched in between LoxP sequences.

### Technical Effects of the Invention

According to the present invention, a transgenic animal other than human that develops a condition of neuropsychiatric disorder can be obtained. A transgenic animal in accordance with the present invention can be a model animal that develops a condition of neuropsychiatric disorder.

Further, a transgenic animal other than human that develops a condition of neuropsychiatric disorder in accordance with the present invention can be used for a screening of a therapeutic agent for neuropsychiatric disorder.

### BRIEF DESCRIPTION OF DRAWINGS

FIG. 1 is a drawing showing a genotype of Gng7^{(+/Cre)}:Hb-egf^{(flox/flox)} (TG mouse).

FIG. 2 shows results of the determination of each genotype of a Gng7^{(+/Cre)} mouse and an Hb-egf^{(flox/flox)} mouse by a PCR method.

FIG. 3 shows LacZ-staining images of the hippocampus region in the brain of an HB-EGF deficient type TG mouse.

FIG. 4 shows LacZ staining images of each hippocampus region in the brains of a Gng7^{(+/Cre)} mouse and an Hb-egf^{(flox/flox)} mouse that are in the control group of an HB-EGF deficient type TG mouse.

FIG. 5 shows results of the observation of each moving distance, which shows the hyperactivity, and each staying time in the center, which shows the anxiety, of an HB-EGF deficient type TG mouse and a Gng7^{(+/Cre)} mouse that is a control mouse.

FIG. 6 shows results of the measurement of each akinesia time, which shows the desperate condition, of an HB-EGF deficient type TG mouse and a Gng7^{(+/Cre)} mouse that is a control mouse.

FIG. 7 shows results of a marble-burying behavior test about the development of obsessive-compulsive disorder of an HB-EGF deficient type TO mouse.

FIG. 8 shows results of the observation of each nest-building of an HB-EGF deficient type TG mouse and a Gng7^{(+/Cre)} mouse that is a control mouse, in order to determine the social behavior and the interest and consideration for the surrounding environment.

FIG. 9 shows results of the observation of the child rearing of each mother of an HB-EGF deficient type TG mouse and a Gng7^{(+/+)}:Hb-egf^{(flox/flox)} mouse that is a control mouse.

FIG. 10 shows results of the examination of each impulsivity and each environmental perception of an HB-EGF deficient type TG mouse and a Gng7^{(+/+)}:Hb-egf^{(flox/flox)} mouse that is a control mouse.

FIG. 11 shows results of the examination of each learned behavior of an HB-EGF deficient type TG mouse and a Gng7^{(+/+)}:Hb-egf^{(flox/flox)} mouse that is a control mouse.

FIG. 12 shows results of the examination of each learned behavior of an HB-EGF deficient type TG mouse and a Gng7^{(+/+)}:Hb-egf^{(flox/flox)} that is a control mouse.

FIG. 13 shows results of the comparison of the expression levels of PSD-95 that binds to each C terminus of a NMDA protein and a glutamate receptor that are involved in the brain function in the hippocampus and controls the localization.

FIG. 14 shows results of the evaluation of the synaptic plasticity in the hippocampal CA1 Region using LTP as an index.

FIG. 15 shows results of the examination of the effects obtained by administering a therapeutic agent to a TG mouse in which neuropsychiatric disorder has been developed.

FIG. 16 shows results of the examination of the effects obtained by administering a therapeutic agent to a TG mouse in which neuropsychiatric disorder has been developed.

FIG. 17 shows results of the examination of the effects obtained by administering a therapeutic agent to a TG mouse in which neuropsychiatric disorder has been developed.

### BEST MODE FOR CARRYING OUT THE INVENTION

An HB-EGF deficient type transgenic animal (TG animal) in the hippocampal neuron region in accordance with the present invention develops a condition of neuropsychiatric disorder. That is, the present invention relates to a transgenic animal in which an HB-EGF gene is deficient, to a production method of a transgenic and to a screening method of a therapeutic agent for neuropsychiatric disorder using the transgenic animal.

### (Heparin-Binding EGF-Like Growth Factor (HB-EGF))

An HB-EGF protein is called as a heparin-binding EGF-like growth factor, and is a growth factor belonging to a EGF family that is a single-pass transmembrane protein and is expressed as a cell-surface membrane protein. The HB-EGF is expressed in various cell strains and shows a growth stimulation activity and a cell migration promotion activity. Further, it is known that the HB-EGF protein is useful as a therapeutic or preventive drug for various heart diseases. However, the role of the HB-EGF gene in the hippocampal neuron region, and the characteristics of the TG animal in which an HB-EGF gene is deficient in the hippocampal neuron region are unclear.

In the present invention, a TG animal (Gng7^{(+/Cre)}:Hb-egf^{(flox/flox)} in which an HB-EGF gene is deficient can be specifically obtained in the hippocampal neuron region (spiny neurons (striatum, and hippocampus)). The TG animal (Gng7^{(+/Cre)}:Hb-egf^{(flox/flox)} in which an HB-EGF gene is specifically deficient in the spiny neurons (striatum, and hippocampus) of the brain can be obtained by crossbreeding a Gng7^{(+/Cre)} animal and an Hb-egf^{(flox/flox)} animal.

### (Gng7^{(+/Cre)} Animal)

A Gng7^{(+/Cre)} animal is a transgenic animal in which Cre protein is expressed in the spiny neurons by using a gene promoter Gng7 that is specifically expressed in the spiny neurons (striatum, and hippocampus). In the Gng7^{(+/Cre)} animal, a gene promoter Gng7 that is specifically expressed in the spiny neurons (striatum, and hippocampus) is arranged at an upstream of the Cre gene. Therefore, the Cre protein is specifically expressed in the spiny neurons (striatum, and hippocampus).

### (Cre Protein)

A Cre protein is an enzyme that recognizes a gene region sandwiched in between LoxP sequences, and excises the gene region between the LoxP sequences. By the Cre protein, a gene region can be excised if the region is sandwiched in between LoxP sequences, therefore, an individual in which a specific gene is deficient can be obtained.

By binding a promoter that functions only in a specific tissue at an upstream of a Cre gene, a Cre protein can be expressed only in the specific tissue. In a Gng7^{(+/Cre)} animal, a gene promoter Gng7 that is specifically expressed in the spiny neurons (striatum, and hippocampus) is sequenced at an upstream of the Cre gene, therefore, the Cre protein is specifically expressed in the spiny neurons (striatum, and hippocampus).

The Cre protein is a protein containing 343 amino acid residues. The amino acid sequence of the Cre protein is shown in SEQ ID NO: 1. In the present invention, even if the amino acid sequence is an amino acid sequence in which one or several (2 to 6) amino acids are deleted, substituted, inserted, or added in the amino acid sequence of SEQ ID NO: 1, as is the case in the Cre protein, if the protein is a protein that exerts a function excising a gene region sandwiched in between LoxP sequences, the protein can be used.

Further, as is the case in the Cre protein, it can be determined whether the protein is a protein that exerts a function excising the gene region sandwiched in between LoxP sequences or not by performing a genetic analysis.

A Cre protein recognizes a gene region sandwiched in between LoxP sequences, and plays a role of excising the gene region between the LoxP sequences. In a Cre protein, loxP recognizes a sequence ATAACTTCGTATA- at the 5′ end and a sequence -IATACGAAGTTAT at the 3′ end of the sequence, and binds the sequences. A Cre protein in which loxP has bound to the sequence attacks the cleavage site of the LoxP sequence, and excises the gene region therebetween.

### (Promoter)

A promoter is a gene sequence that induces transcription. A promoter is expressed cell-strain specifically, or tissue-specifically, and the transcription is induced by binding an external signal or a factor to the promoter.

A promoter to be used in the present invention is preferably a promoter that is specifically expressed in the hippocampal neuron region. Particularly, it is preferable to use a promoter that is specifically expressed in the spiny neurons (striatum, and hippocampus) of the brain.

A promoter that can be used in the present invention is a Gng7 promoter, a CamK II promoter, or an Emx1 promoter. In particular, a Gng7 promoter is a promoter that is specifically expressed particularly in the spiny neurons (striatum, and hippocampus) of the brain, therefore, is preferable.

### Hb-egf^{flox/flox)} Animal)

In an Hb-egf^{(flox/flox)} animal, a loxP gene region is sequenced before and after the HB-EGF gene. The sequence of loxP is a sequence consisting of 34 bases, and the gene sequence is shown in SEQ ID NO: 2. In the present invention, even if the base sequence is a base sequence in which one or several (2 to 6) bases of the loxP base sequence are deleted, substituted, inserted, or added, as is the case in the loxP sequence, if the base sequence contains a binding site and a cleavage site of the Cre protein, it can be used.

In an Hb-egf^{(flox/flox)} animal, if the Cre protein is not present, the HB-EGF is expressed as usual. However, if the Cre protein is expressed in an Hb-egf^{(flox/flox)} animal, an HB-EGF gene is excised, therefore, the expression of the HB-EGF protein is suppressed.

### (LacZ)

In an Hb-egf^{(flox/flox)} animal, a gene that expresses a LacZ protein is sequenced at a downstream of the Hg-egf gene. In a tissue in which Cre is expressed, by the Cre protein, an HB-EGF gene sequence between the loxP sequences is removed, and LacZ that has arranged at a downstream of the HB-EGF gene is expressed instead. That is, a part in which LacZ has expressed becomes a part in which the expression of the HB-EGF gene has suppressed by the presence of the Cre protein. LacZ plays a role as a tracer of the deficiency of the HB-EGF gene.

### (Gng7^{(+/Cre)}:Hb-egf^{(flox/flox)} Animal)

A TG animal (Gng7^{(+/Cre)}:Hb-egf^{(flox/flox)}) in which an HB-EGF gene is deficient of the present invention can be obtained by crossbreeding (re)+/C a Gng7^{(+/Cre)} animal and an Hb-egf^{(flox/flox)} animal, and by selecting an animal that has both genotypes of the Gng7^{(+/Cre)} animal and the Hb-egf^{(flox/flox)} animal.

A TG animal (Gng7^{(+/Cre)}:Hb-egf^{(flox/flox)} that is obtained by crossbreeding a Gng7^{(+/Cre)} animal and an Hb-egf^{(flox/flox)} animal has both genotypes of the above-mentioned Gng7^{(+/Cre)} animal and Hb-egf^{(flox/flox)} animal. Therefore, in the hippocampal neuron region of the TG animal (Gng7^{(+/Cre)}:Hb-egf^{(flox/flox)}, a Cre protein is produced. Further, before and after the Hg-egf gene of the TG animal (Gng7^{(+/Cre)}:Hb-egf^{(flox/flox)}, loxP is sequenced.

A Cre protein recognizes a gene region sandwiched in between LoxP sequences and excises the region, therefore, the Hg-egf gene of the TG animal (Gng7^{(+/Cre)}:Hb-egf^{(flox/flox)} is deleted by the Cre protein. Therefore, in the hippocampal neuron region of the TG animal (Gng7^{(+/Cre)}:Hb-egf^{(flox/flox)}), an HB-EGF gene becomes deficient.

A transgenic animal in which a specific gene is made deficient is an animal that cannot produce the gene product as a result of artificially modifying a specific gene. A transgenic animal in which a specific gene is made deficient is useful for examining a function of the specific gene, or for screening a therapeutic agent for a condition that is developed by deleting the specific gene.

In the present invention, a promoter Gng7 is arranged at an upstream of the Cre gene, therefore, a Cre protein can be expressed only in the hippocampal neuron region. Therefore, in the hippocampal neuron region, an HB-EGF gene can be specifically made deficient.

In the present invention, the HB-EGF gene can be made deficient only in the hippocampal neuron region, therefore, the HB-EGF gene can be normally expressed in other tissues of the transgenic The HB-EGF gene has important functions even in a tissue other than that in the hippocampal neuron region, and thus, in the present invention, the HB-EGF gene is normally expressed in other tissues, therefore, the fatal impact cannot be provided to the growth of the transgenic animal. That is, in the present invention, embryonic lethality of a mouse can be prevented.

Further, in the present invention, there is no need to consider the influence of the deficiency of the HB-EGF gene in other tissues, therefore, the function of the HB-EGF gene in the hippocampal neuron region can be clarified.

Further, in the present invention, only in the spiny neurons (striatum, and hippocampus), the HB-EGF gene is made deficient, therefore, the functional analysis of the deficient gene is readily performed, and the screening of a therapeutic agent can also be effectively performed.

In the present invention, it was clear by Examples that in the hippocampal neuron region, a transgenic animal in which an HB-EGF gene was made deficient developed a condition of neuropsychiatric disorder. As long as the neuropsychiatric disorder is a disease in which a Hg-egf gene is involved, it is not particularly limited, for example, examples of the neuropsychiatric disorder include depression, schizophrenia, obsessive-compulsive disorder, attention deficit hyperactivity disorder, pervasive developmental disorder containing Asperger's syndrome, Alzheimer disease, learning disability, and long-term memory impairment.

A transgenic animal other than human in which an HB-EGF gene is made deficient in accordance with the present invention can be used without any particular limitations as long as the transgenic animal is an animal other than human, for example, a mammal such as a mouse, a rat, a monkey, a bovine, and a canine, can be used. Among them, a transgenic animal is preferably a mouse.

A mouse has an advantage in growing rapidly, being a small and thus being easy to deal with. Further, a mouse develops a symptom that is close to the symptom of the neuropsychiatric disorder of human, therefore, can be a model animal used for a screening of a therapeutic agent.

NR1, PSD-95, and NR2B are important proteins involved in learning, or psychiatric disorder. In a TG animal (Gng7^{(+/Cre)}:Hb-egf^{(flox/flox)}) of the present invention, the levels of the expression of NR1, PSD-95, and NR2B are decreased.

That is, in a TG animal (Gng7^{(+/Cre)}:Hb-egf^{(flox/flox)}), the expression of the HB-EGF protein is suppressed, and thus the levels of the expression of these proteins may be decreased. In the HB-EGF deficient type TG mouse in accordance with the present invention, the decrease of the expression of a NMDA receptor is generated via PSD-95, and thus it is suggested that the decrease becomes a cause showing the pathology that relates to the cognitive function relating to the neuronal plasticity in the hippocampus.

The present invention relates to a method for producing a transgenic animal in which an HB-EGF gene is made deficient and a condition of neuropsychiatric disorder is developed. A production method in accordance with the present invention is method for obtaining a transgenic animal in which an HB-EGF gene is deficient by crossbreeding a first transgenic animal other than human that contains a gene promoter being specifically expressed in the hippocampal neuron region and a Cre gene sequence, and a second transgenic animal other than human that contains an HB-EGF gene sandwiched in between LoxP sequences.

By crossbreeding a first transgenic animal other than human that contains a gene promoter that is specifically expressed in the hippocampal neuron region, and a Cre gene sequence, and a second transgenic animal other than human that contains an HB-EGF gene sequence sandwiched in between LoxP sequences, a Gng7^{(+/Cre)}/Hb-egf^{(flox/flox)} animal is generated with a probability of 50% in the theoretical value. An animal containing a genotype of Gng7^{(+/Cre)}/Hb-egf^{(flox/flox)} is a transgenic animal in which an HB-EGF gene is deficient, and is a transgenic animal that is targeted in the present invention. In a transgenic animal that is generated by crossbreeding a first transgenic animal and a second transgenic animal, a targeted transgenic animal in which an HB-EGF gene is deficient, and a non-targeted transgenic animal in which an HB-EGF gene is not deficient are mixed. Therefore, from the generated transgenic animals, in order to obtain the targeted transgenic animal in which an HB-EGF gene is deficient, the genotype is analyzed by a PCR method, and the targeted transgenic animal in which an HB-EGF gene is deficient is selected. By analyzing and selecting the genotype, a Gng7^{(+/Cre)}/Hb-egf^{(flox/flox)} animal in which an HB-EGF gene is deficient can be obtained.

By crossbreeding two types of the transgenic animals, that is, a Gng7^{(+/Cre)} animal and an Hb-egf^{(flox/flox)} animal, a conditional transgenic animal can be produced. The conditional transgenic animal obtained by crossbreeding two types of mice does not cause embryonic lethality even an HB-EGF gene that is largely involved in a biological phenomenon is made deficient, therefore, is useful in examining the relation between the HB-EGF gene and the neuropsychiatric disorder.

A conditional transgenic animal is referred to an animal, in which a specific gene is made deficient in a specific tissue at a specific time. In a conditional transgenic animal, a specific gene can be made deficient tissue-specifically or time-specifically, therefore, neuropsychiatric disorder in which a specific gene is involved can be clearly specified.

The present invention relates to a method for a screening a therapeutic agent for neuropsychiatric disorder. The screening method in accordance with the present invention is a method in which a test substance is administered to a transgenic animal other than human in which an HB-EGF gene is deficient and a symptom of neuropsychiatric disorder has developed, and an effect of the improvement of the condition of neuropsychiatric disorder is examined. A transgenic animal in which an HB-EGF gene is deficient in accordance with the present invention develops neuropsychiatric disorder. To the transgenic animal, a test substance is administered, and by measuring the degree of the development of neuropsychiatric disorder and the degree of the progression of the disorder, thus a screening can be readily performed. Further, also by using an expression of other genes that is activated by administering a test substance as an index, a screening of a therapeutic agent can be performed.

Further, the transgenic animal other than human in which a symptom of neuropsychiatric disorder has developed in accordance with the present invention can be used not only for a screening, but also for the study or clinical research as a model animal for examining the relation between the HB-EGF gene and the neuropsychiatric disorder.

The dosage form of a therapeutic agent can be selected from the existing dosage forms, and may be any one of liquid formulation, solid formulation, and vapor formulation. In the therapeutic agent, known pharmaceutically acceptable additive agents can be added. The dosage of the therapeutic agent varies depending on the kind of active ingredient, the administration method, the symptom, and the kind of a target to be administered, for example, if the mouse weighs around 20 to 30 g, the dosage is appropriately 0.05 to 30 mg/kg when administered intraperitoneally.

As the administration method of a therapeutic agent, a method of oral administration or parenteral administration can be mentioned. In the case of oral administration, it is preferable that tablet, granules, or liquid is administered once or several times a day. In the case of parenteral administration, it is preferable that liquid formulation containing a therapeutic agent is administered in a living body by perfusion or intravenous injection. Further, in the case of parenteral administration, the therapeutic agent may be directly administered into the abdominal cavity or the cerebral ventricle.

### Example 1

### Production of Gng7^{(+/Cre)} Mouse

As a Gng7^{(+/Cre)} mouse, a mouse that has deposited to RIKEN and is a known mouse was used. The production method of the Gng7^{(+/Cre)} mouse is described in PNAS, 100 (6)3221 to 3226, 2003.

### Production of Hb-egf^{(flox/flox)} Mouse

As an Hb-egf^{(flox/flox)} mouse, a known mouse was used. The production method of the Gng7^{(+/Cre)} mouse is described in Plos One Vol 4 Issue 1e4157. January, 2009.

### Production of HB-EGF Deficient Type Transgenic Mouse

By crossbreeding a Gng7^{(+/Cre)} mouse and an Hb-egf^{(flox/flox)} mouse, the second generation transgenic mouse was obtained. A genome extracted from part of the ear (a diameter of 2 mm) of the second generation transgenic mouse was determined by using a PCR method. By a PCR method, the second generation transgenic mouse that contains both genes of the Gng7^{(+/Cre)} mouse and the Hb-egf^{(flox/flox)} mouse was selected, and an HB-EGF deficient type transgenic mouse was obtained.

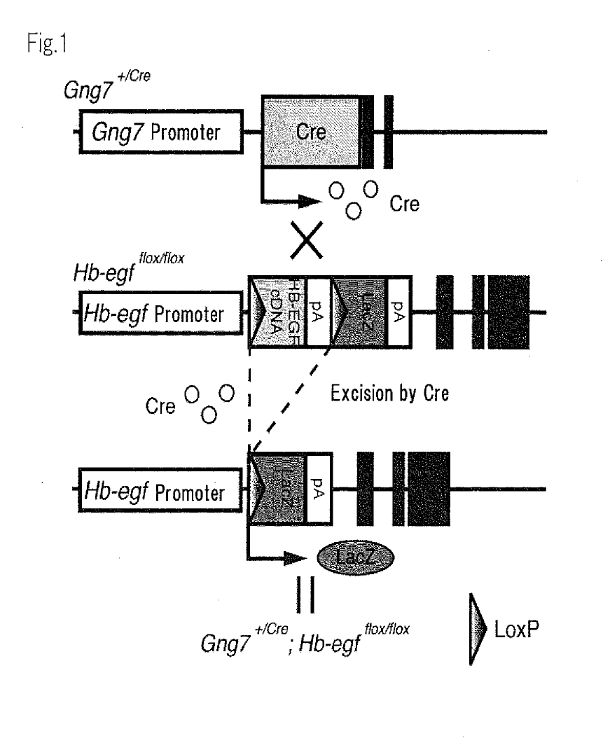

FIG. 1 is a schematic drawing showing a genotype of Gng7^{(+/Cre)}:Hb-egf^{(flox/flox)} (TG mouse). A Gng7^{(+/Cre)} mouse contains a Gng7 promoter and a Cre gene sequence. A Hb-egf^{(flox/flox)} mouse contains an HB-EGF gene sequence sandwiched in between LoxP sequences. By crossbreeding the Gng7^{(+/Cre)} mouse and the Hb-egf^{(flox/flox)} mouse, a Gng7^{(+/Cre)}:Hb-egf^{(flox/flox)} animal can be obtained.

A Gng7 promoter is specifically expressed in the hippocampal neuron region, therefore, the Cre protein is expressed only in the hippocampal neuron region. The Cre protein specifically expressed in the hippocampal neuron region recognizes a LoxP sequence and excises an HB-EGF gene sandwiched in between LoxP sequences. A gene sequence after the HB-EGF gene was excised is shown in the bottom drawing of FIG. 1. A LacZ gene is arranged at a downstream of the HB-EGF gene, therefore, LacZ is expressed by an Hb-egf promoter after the HB-EGF gene was excised. That is, LacZ plays a role as a tracer of the deficiency of the HB-EGF gene.

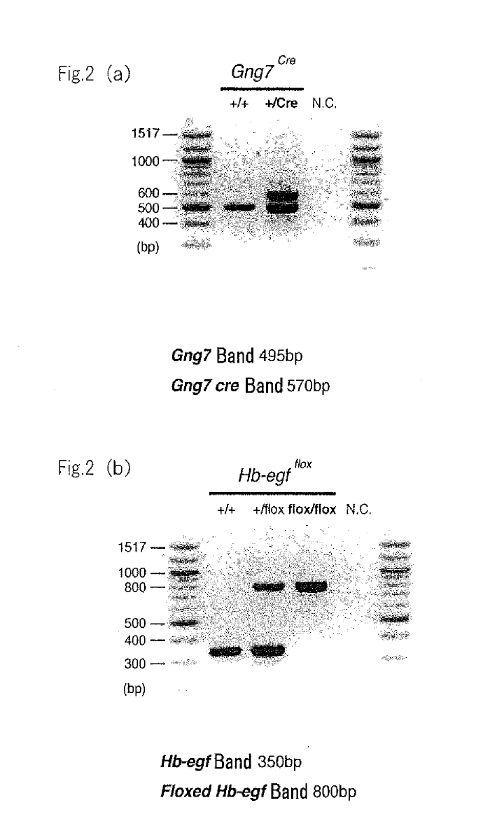

FIG. 2 shows results of the determination of each genotype of a Gng7^{(+/Cre)} mouse and an Hb-egf^{(flox/flox)} mouse by a PCR method.

FIG. 2(a) is a picture showing the genotype of a Gng7^{(+/Cre)} mouse as a result of PCR. As a primer, a primer (G7NR) described in SEQ ID NO: 3, a primer (CreMetR) described in SEQ ID NO: 4, and a primer (KpEco) described in SEQ ID NO: 5 were used. All the primers were diluted with sterile water so as to be a solution with 10 pmol/μl and used. By mixing 0.4 μl of G7NR primer, 2.4 μl of CreMetR primer, 0.8 μl of KpEco primer, 10 μl of 2xMangoMix containing reaction buffer solution, enzyme and dNTP mix, 1 μl of genomic DNA extract, and 5.4 μl of sterile water, total 20 μl of the reaction solution was prepared. Next, the reaction solution was subjected to PCR to amplify the base sequence in a target region. DNA denaturation was performed with the PCR reaction conditions of 95° C. and 120 seconds, and then the DNA denaturation was performed with the conditions of 95° C. and 30 seconds, 60° C. and 20 seconds, and 72° C. and 30 seconds, and thus the primers were annealed. This series of reactions was performed 35 cycles. After the final cycle, the reaction solution was incubated at 72° C. for 300 seconds, and stored at 4° C. The electrophoresis of the PCR reaction solution was performed by using 2% agarose gel and a TAE buffer solution at 170V for 30 minutes. As a result, in the lane (+/Cre) to which an intended PCR product was poured, a Gng7 band at the position of 495 bp, and a Gng7 Cre band at the position of 570 by were confirmed.

FIG. 2(b) is a picture showing the genotype of an Hb-egf^{(flox/flox)} mouse as a result of PCR. As a primer, a primer (Neo-1) described in SEQ ID NO: 6, a primer (Neo-2) described in SEQ ID NO: 7, a primer (HBint1) described in SEQ ID NO: 8, and a primer (5-552) described in SEQ ID NO: 9 were used. All the primers were diluted with sterile water so as to be a solution with 10 pmol/μl and used. By mixing 0.8 μl of Neo-1 primer, 0.8 μl of Neo-2 primer, 0.8 μl of HBint1 primer, 0.8 μl of 5-5S2 primer, 10 μl of 2xMangoMix containing reaction buffer solution, enzyme and dNTP mix, 1 μl of genomic DNA extract, and 5.8 μl of sterile water, total 20 μl of the reaction solution was prepared. Next, the reaction solution was subjected to PCR to amplify the base sequence in a target region. DNA denaturation was performed with the PCR reaction conditions of 94° C. and 180 seconds, and then the DNA denaturation was performed with the conditions of 94° C. and 30 seconds, 57° C. and 30 seconds, and 72° C. and 60 seconds, and thus the primers were annealed. This series of reactions was performed 35 cycles. After the final cycle, the reaction solution was incubated at 72° C. for 300 seconds, and stored at 4° C. The electrophoresis of PCR reaction solution was performed by using 2% agarose gel and a TAE buffer solution at 170V for 30 minutes.

As a result, in the lane (flox/flox) to which an intended PCR product was poured, a Floxed-Hg-egf band at the position of 800 bp was confirmed.

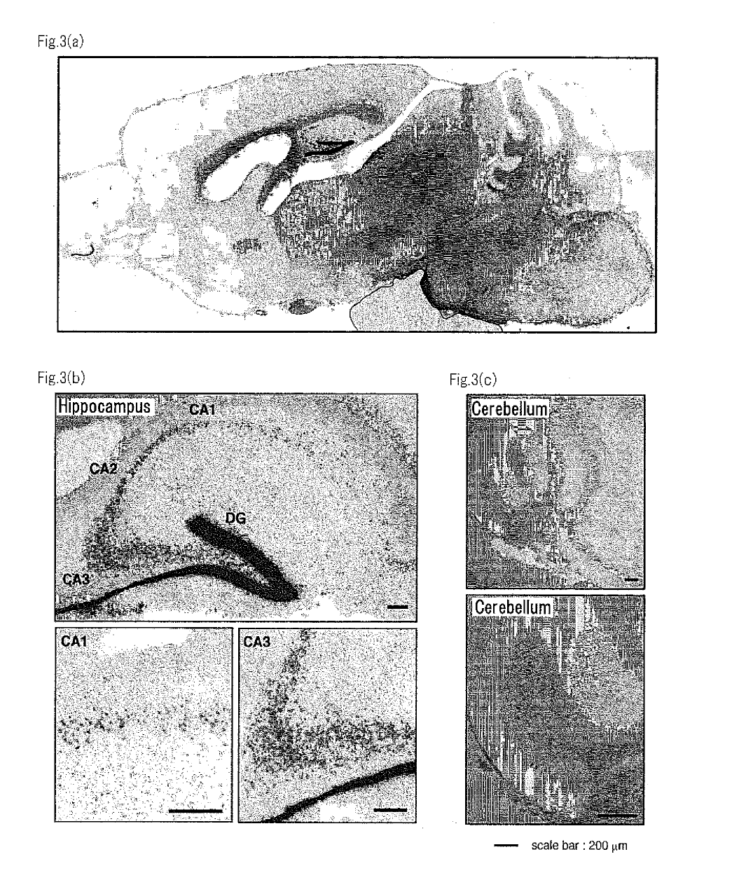

FIG. 3 shows LacZ-staining images of the hippocampus region in the brain of an HB-EGF deficient type TG mouse.

FIG. 3(a) shows an image in the periphery of the hippocampus region. The central part of FIG. 3(a) is the hippocampus, and the right side part is the cerebellum. In FIG. 3(a), the staining of a LacZ protein cannot be shown closely, therefore, the hippocampus region and the cerebellum region are further enlarged and shown in FIG. 3(b) and FIG. 3(c).

FIG. 3(b) shows an image further enlarged the hippocampus region (the upper picture of FIG. 3(b)), and images further enlarged the CA1 region (the left-lower picture of FIG. 3(b)) and the CA3 region (the right-lower picture of FIG. 3(b)) of the hippocampus. In FIG. 3(b), it is understood that LacZ is specifically expressed in the DG region (dentate gyrus) of the central part of the hippocampus, and is stained most densely. Further, it is understood that also in the CA3 region of the hippocampus, LacZ is expressed relatively largely.

FIG. 3(c) shows images in the periphery of the cerebellum. The upper image of FIG. 3(c) shows an enlarged image in the periphery of the cerebellar granule cells, and the lower image shows an image further enlarged the periphery of cerebellar granule cells. As shown in FIG. 3(c) it is understood that the LacZ protein is expressed in the periphery of the cerebellar granule cells.

As shown in FIG. 3, the LacZ protein is specifically expressed in the central region (dentate gyrus) of the hippocampus in the brain. Therefore, it was found that the deficiency of an HB-EGF gene was caused in the central region (dentate gyrus) of the hippocampus in the brain.

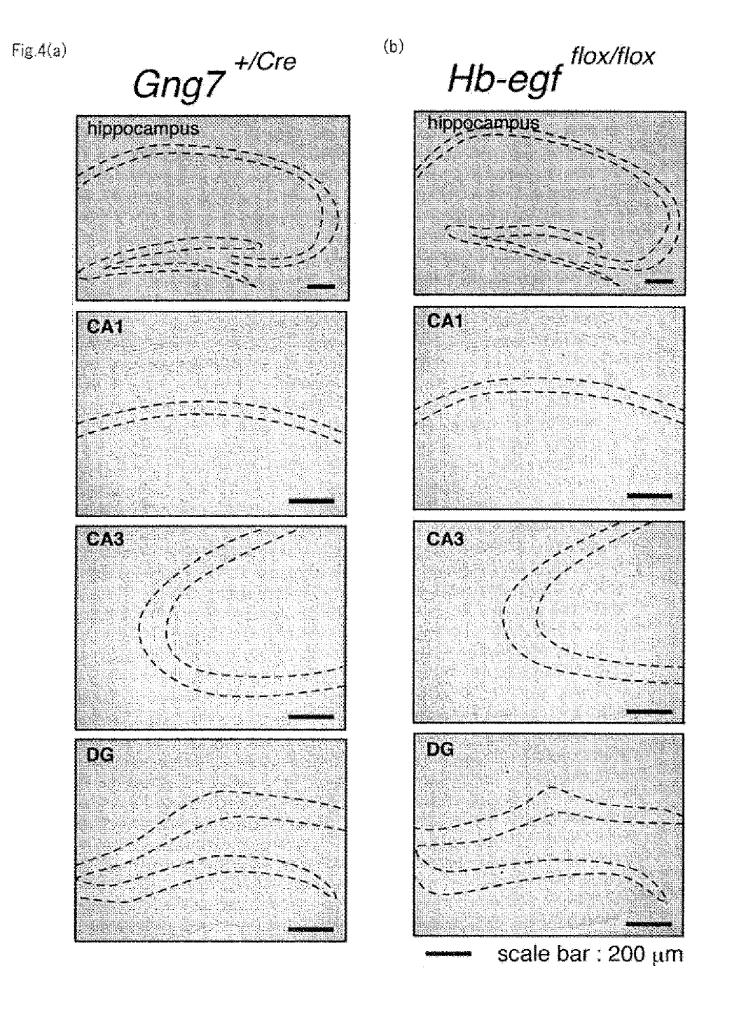

FIG. 4 shows LacZ staining images of the hippocampus regions in the brains of a Gng7^{(+/Cre)} mouse and an Hb-egf^{(flox/flox)} mouse that are in the control group of the HB-EGF deficient type TG mouse. In the Gng7^{(+/Cre)} mouse, an HB-EGF gene is not excised, therefore, the expression of the LacZ was not observed.

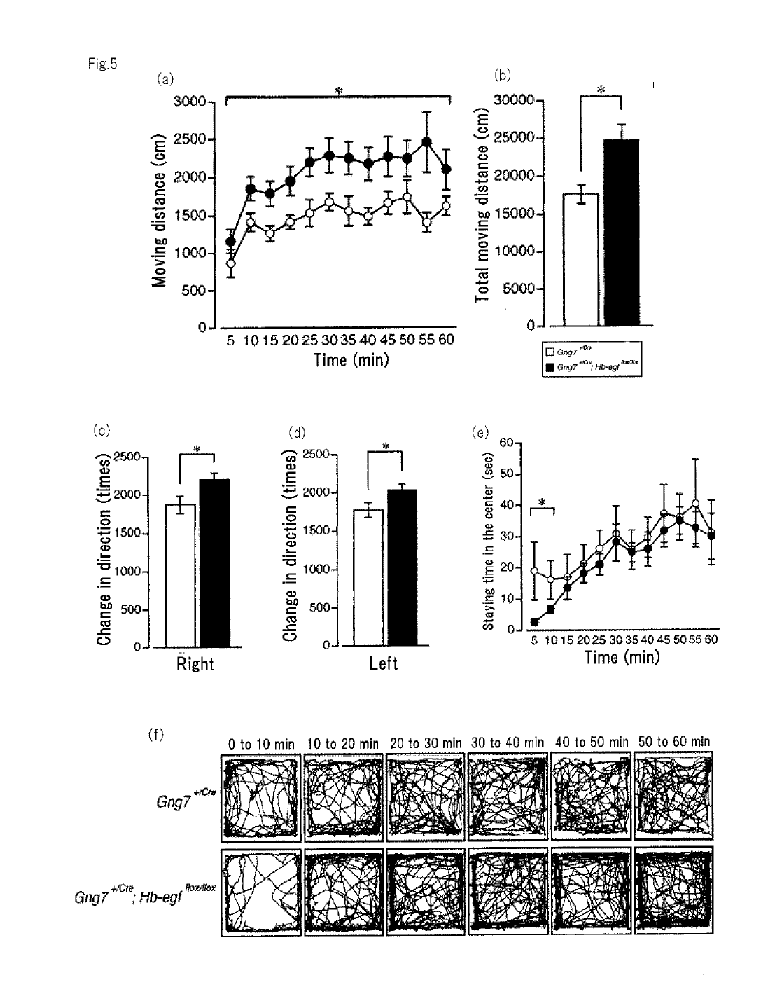

FIG. 5 shows results of the observation of each behavior of an HB-EGF deficient type TG mouse and a Gng7^{(+/Cre)} mouse that is a control mouse. From the control group of the TG mouse, a Gng7^{(+/Cre)} mouse was used.

Each mouse was housed in a cage with a field of square shape of 70 cm by 70 cm by 30 cm in depth and with the lighting of around 50 lux, the acting time in 30 minutes, the moving velocity, the moving distance, the staying time in the center, the number of right and left turnings, the number of changes in right or left direction, and the movement locus were tracked by a video tracking camera (manufactured by Muromachi Kikai Co., Ltd.), and analyzed on a computer.

FIG. 5(a) shows results of the moving distance that each mouse moved in a cage every passage of time. It is understood that the acting amount of the TG mouse each time was significantly increased as compared with the Gng7^{(+/Cre)} mouse.

FIG. 5(b) shows results of the total moving distance that each mouse moved in a cage. It is understood that the total acting amount of the TG mouse was significantly increased as compared with the Gng7^{(+/Cre)} mouse.

FIG. 5(c) shows results of the number of changes in right direction while each mouse was moving in a cage. It is understood that the number of changes in right direction of the TG mouse was significantly increased as compared with the Gng7^{(+/Cre)} mouse.

FIG. 5(d) shows results of the number of changes in left direction while each mouse was moving in a cage. It is understood that the number of changes in left direction of the TG mouse was significantly increased as compared with the Gng7^{(+/Cre)} mouse.

FIG. 5(e) shows results of the staying time that each mouse stayed in the central part of a cage. It is understood that the TG mouse did not move to the central region in early time (in 0 to 10 minutes), and the time staying in the central region was short. The decrease of the moving to the center in early time (in 0 to 10 minutes) shows a symptom of the anxiety enhancement.

FIG. 5(f) shows results of the movement locus that each mouse moved in a field. The TG mouse did not move to the central region in early time (in 0 to 10 minutes), and the increase of the moving amount was observed in the marginal region of the field.

From the results of FIGS. 5(a) to 5(f), it was understood that the moving distance of the HB-EGF deficient type mouse was longer than that of the mouse in which the HB-FGF was not deficient. Further, it was also understood that the number of changes in direction of the HB-EGF deficient type mouse was larger than that of the mouse in which HB-EGF was not deficient. This is considered to be an influence of the hyperactivity.

Further, it was understood that the time staying in the central part of a cage of the HB-EGF deficient type mouse was shorter in early time (in 0 to 10 minutes). Therefore, it was understood that the HB-EGF deficient type mouse has low ability to adapt to an environmental change, and the nature easily feeling anxiety.

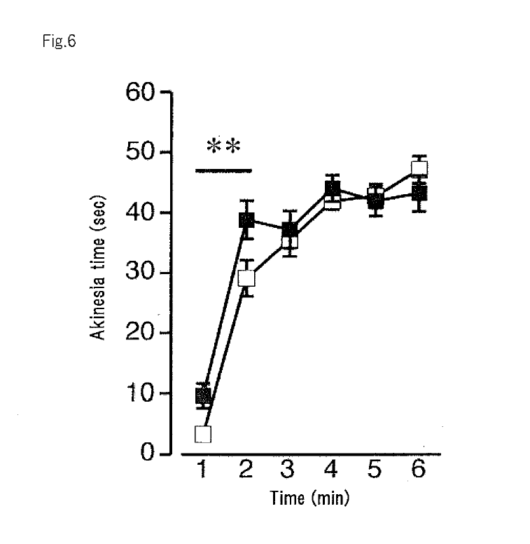

FIG. 6 shows results of the measurement of each akinesia time, which shows the desperate condition, of an HB-EGF deficient type TG mouse and a Gng7^{(+/Cre)} mouse that is a control mouse. Into an acrylic column with a height of 30 cm and a diameter of 20 cm, water at 25±1° C. was added up to the 20 cm height of the acrylic column, and the akinesia time of each mouse on the water surface was measured for 6 minutes at an interval of one minute. In the HB-EGF deficient type TG mouse, the extension of akinesia time was observed in early time (in 1 to 2 minutes) as compared with the Gng7^{(+/Cre)} mouse. This shows that in the HB-EGF deficient type TG mouse, the transition to the desperate condition is quick. That is, it is understood that in the HB-EGF deficient type TG mouse, the threshold value until a depression-like symptom is developed is low.

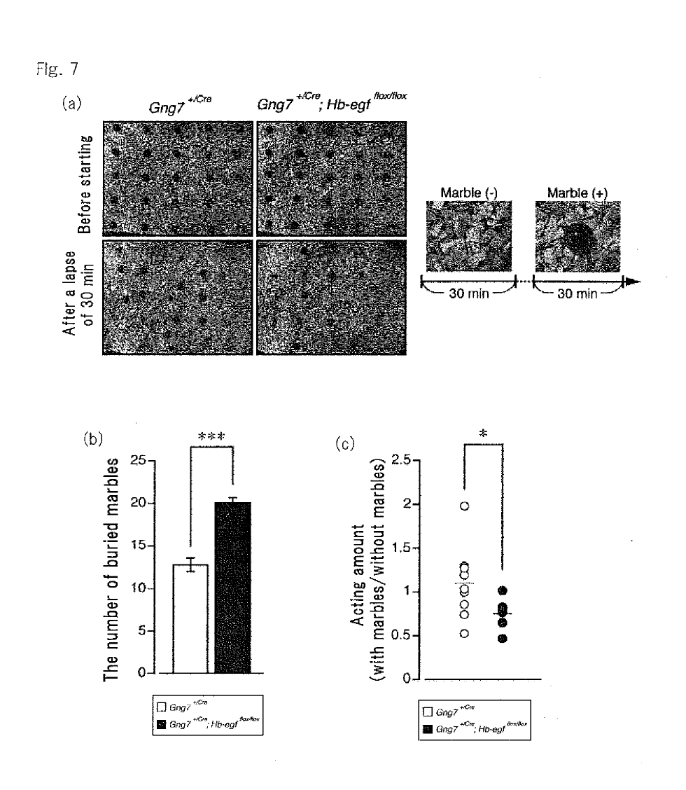

FIG. 7 shows results of the examination of the development of obsessive-compulsive disorder in the HB-EGF deficient type TG mouse. The obsessive-compulsive disorder was evaluated by a marble-burying behavior test.

The behavior of a mouse, that is, the mouse tries to bury harmless marbles with the bedding, is similar to the abnormal behavior of a patient who developed obsessive-compulsive disorder, therefore, the behavior, that is, the mouse tries to bury harmless marbles with the bedding is recognized to be used as an anxiety-like behavior relating to obsessive-compulsive disorder.

The marble-burying behavior test was carried out in accordance with the following processes. First, in a cage in which bedding was carpeted in around 5 cm depth, 25 blue-colored marbles with 17 mm were equally arranged, and the marble-burying behavior of each mouse were measured for 30 minutes. After a lapse of 30 minutes, when observed from straight above, the marbles those were buried two-thirds or more thereof with bedding were counted. Further, the acting amount of burying marbles was determined by SCANET. In the marble-burying behavior test, the more the number of marbles that buried after the test, the more the anxiety-like behavior enhanced.

FIG. 7(a) shows results of the marbles before and after each marble-burying behavior test of the HB-EGF deficient type TG mouse and the Gng7^{(+/Cre)} mouse that is a control mouse. As is seen from the results in FIG. 6(a) after a lapse of 30 minutes, the HB-EGF deficient type TG mouse buried more marbles.

FIG. 7(b) shows a graph for the results of the test of FIG. 7(a), that is, shows the number of the buried marbles. It is understood that the HB-EGF deficient type TG mouse buried more marbles as compared with the mouse in which HB-EGF is not deficient.

FIG. 7(c) shows the results of the comparison of the hyperactivity of each mouse in the case of presenting marbles and in the case of not presenting marbles. When the hyperactivity of each mouse between in the case of presenting marbles and in the case of not presenting marbles was not different, the ratio of the acting amount is 1. On the other hand, when the mouse showed more hyperactivity in the case of not presenting marbles, the value of the acting amount is less than 1.

As shown in FIG. 7(c), it is understood that the HB-EGF deficient type TG mouse has smaller ratio of the acting amount. From the above, it is understood that the hyperactivity of the HB-EGF deficient type TG mouse is suppressed in the case of presenting marbles. That is, it is understood that the HB-EGF deficient type TG mouse responded excessively to the marbles, therefore, the hyperactivity is suppressed.

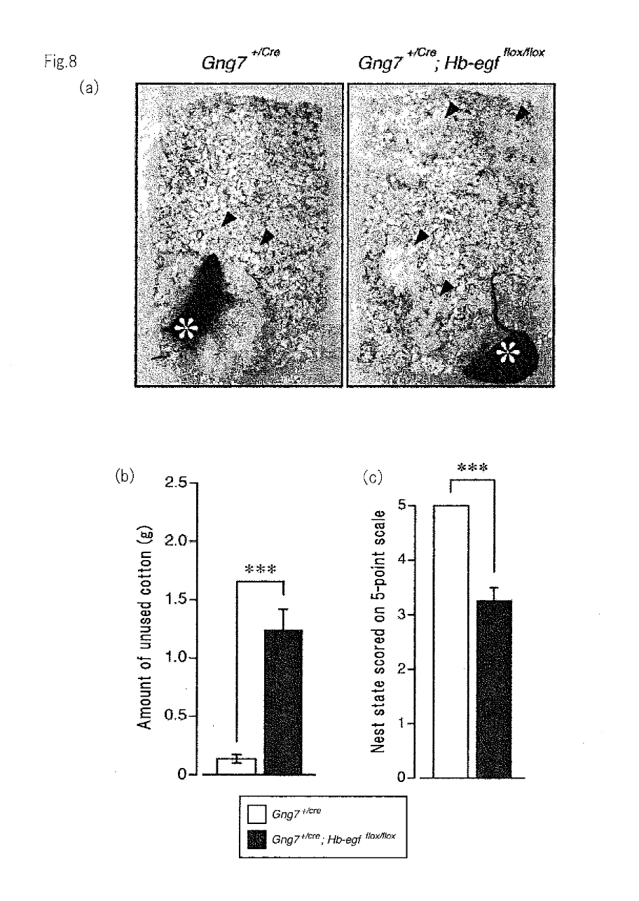

FIG. 8 shows results of the observation of each nest-building of the HB-EGF deficient type TG mouse and the Gng7^{(+/Cre)} mouse that is a control mouse. By observing the nest-building, the social behavior depending on the hippocampus, and the interest and consideration for the surrounding environment can be measured.

The nest-building test was carried out in accordance with the following processes. First, from one hour before the darkness period (19:00) of the light and darkness cycle (light period 8:00 to 20:00/darkness period 20:00 to 8:00), each mouse was reared alone in a new cage, and provided with Nestlet (2.5 g) that is a material for the nest-building. During the darkness period 20:00 to 8:00, each mouse acted freely, in the next morning the nest shape that was made by each mouse was scored on 5-point scale, and the unused Nestlet amount was measured. The score was determined by the following criteria.

1: 90% of the Nestlet was unused.

2: the Nestlet was slightly used, however, 50 to 90% of the Nestlet was left unused.

3: 50 to 90% of the Nestlet was torn off finely, and the shape was not maintained as the nest.

4: 90% or more of the Nestlet was torn off finely, and used as a material of the nest, however, the shape of the nest showed the height lower than that of the mouse and was flat.

5: 90% or more of the Nestlet was torn off finely, and used as a material of the nest, the nest was dug into a crater-like shape, and the (almost) perfect nest that buries the whole body of the mouse when viewed from the side was confirmed.

FIG. 8(a) shows results of each nest-building of the HB-EGF deficient type TG mouse and the Gng7^{(+/Cre)} mouse that is a control mouse. The Gng7^{(+/Cre)} mouse in which HB-EGF is not deficient could make the nest that was taller than the mouse itself, however, the HB-EGF deficient type TG mouse could not make the nest at all.

FIG. 8(b) shows results of the amount of the cotton that was not used for the nest-building in the cotton that is to be used by each mouse for the nest-building. In the mouse in which HB-EGF is not deficient, the cotton that was not used is around 0.3 g, however, in the HB-EGF deficient type TG mouse, the cotton that was not used is around 1.3 g. From the above, it was understood that the HB-EGF deficient type TG mouse could not collect the cotton in one place.

FIG. 8(c) shows results of the evaluation on 5-point scale for the state of the nest that was made by each mouse. In the mouse in which HB-EGF is not deficient, the nest taller than the mouse itself was made, therefore, the scale is 5, however, in the HB-EGF deficient type TG mouse, the flat cotton floor could be only made, and thus the scale is 3.

From the above results, it is understood that in the HB-EGF deficient type TG mouse, the surrounding environmental consideration is not sufficient, and the social nature is extremely decreased.

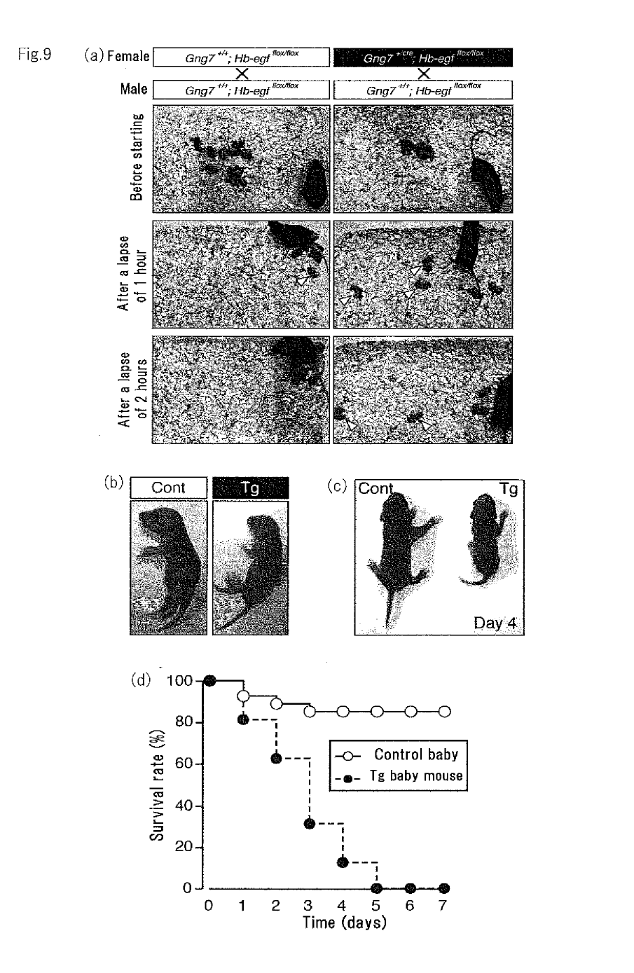

FIG. 9 shows results of the observation of the child rearing of each mother of the HB-EGF deficient type TG mouse and the Gng7^{(+/+)}:Hb-egf^{(flox/flox)} mouse that is a control mouse.

In order to measure the maternal behavior, the breedcage of one female Gng7^{+/cre};Hb-egf^{(flox/flox)} mouse and one male Hb-egf^{flox/flox} mouse was referred to as a Transgenic cage, and the breedcage of one female Hb-egf^{flox/flox} mouse and one male Hb-egf^{flox/flox} mouse referred to as a Control cage, and thus each mouse was bred. After the pregnancy of each female mouse was confirmed, each male mouse was removed, and the solo maternal behavior of each female mouse was compared. The presence or absence of the delivery of each pregnant mouse was observed twice a day at 8:00 and 19:50 without adding stress. In the case that the delivery was not confirmed on the previous night but was confirmed in the morning, the age of the baby mouse sets zero day old. Further, the total number of baby mice at zero day old sets 100%, and the survival rate was measured once a day for total 7 days. Further, one day after the delivery, baby mice and the mother mouse that had delivered the baby mice were arranged in the center of a new cage (0 hour), it was observed in 1 hour and 2 hours whether the mother mouse carried the baby mice to the place where the mother mouse had been decided to be their nest and provided breast milk or not. In order to confirm the feeding, it was observed after a lapse of 2 hours whether the breast milk was confirmed in the abdomen of each baby mouse.

FIG. 9(a) shows results of the observation of the behavior of each mother mouse from collecting the baby mice that had been arranged in the center up to starting the nursing. The delivered female Gng7^{(+/+)}:Hb-egf^{(flox/flox)} mouse in which the HB-EGF is not deficient and that is in a control group can collect the baby mice that had been arranged in the center to one corner in around one hour. On the other hand, the HB-EGF deficient type TG mouse could not collect the baby mice to one place even after a lapse of 2 hours.

FIG. 9(b) shows results of the observation of the abdomen of each baby mouse that was nursed by the mother mouse in which HB-EGF is not deficient and the HB-EGF deficient type TG mother mouse. The baby mouse that had been nursed by the mother mouse in which HB-EGF is not deficient had bulge in the abdomen because the breast milk had reached. On the other hand, the baby mouse that had been nursed by the HB-EGF deficient type TG mother mouse did not have bulge in the abdomen, and it was understood that the baby mouse had not given the breast milk.

FIG. 9(c) shows results of the observation of the baby mouse that was nursed for 4 days under each mouse. It is understood that the baby mouse that was nursed by the HB-EGF deficient type TG mother mouse is smaller and has delayed the development as compared with the baby mouse that was nursed by the mother mouse in which HB-EGF is not deficient.

FIG. 9(d) shows results of the survival rate of the baby mice nursed under each mouse. The survival rate of the baby mice that were nursed by the mother mouse in which HB-EGF was not deficient was 90% or more even after a lapse of 7 days, on the other hand, the survival rate of the baby mice that were nursed by the HB-EGF deficient type TG mother mouse had been falling and became 0% after a lapse of 7 days.

From the above results, it was understood that the HB-EOF deficient type TO mother mouse could not nurse the baby mice well, and showed a nursing neglect-like behavior.

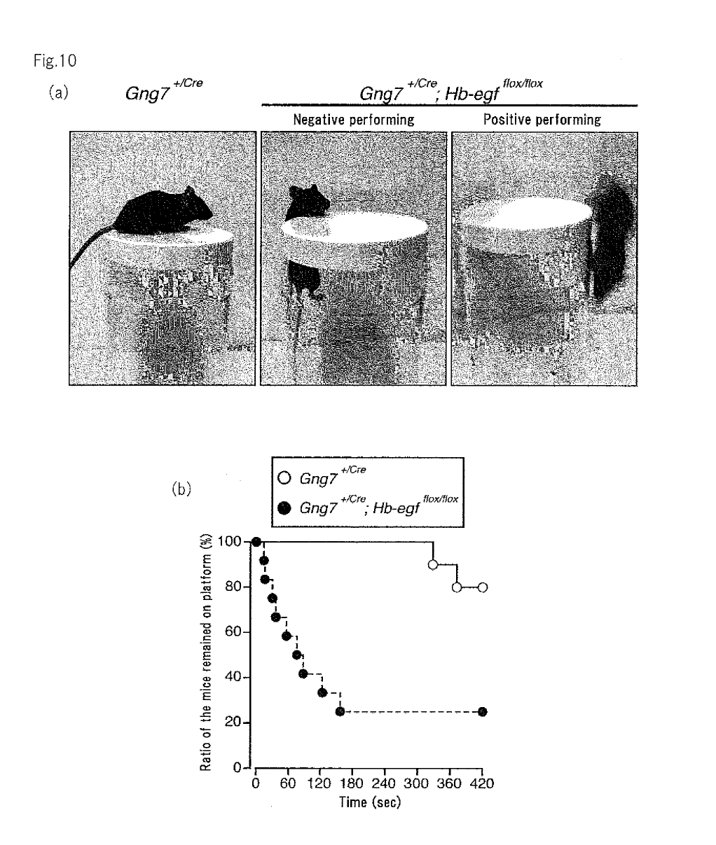

FIG. 10 shows results of the examination of each impulsivity and each environmental perception of the HB-EGF deficient type TG mouse and the Gng7^{(+/Cre)}:Hb-egf^{(flox/flox)} mouse that is a control mouse. As for the impulsivity and environmental perception of the mouse, the state of each mouse when the mouse was placed on a cylindrical platform was observed and evaluated.

Each mouse was arranged on a column having a height of 20 cm and a diameter of 10 cm, and the time course of the descending from the top of the platform to the floor was observed for 7 minutes, and the ratio of the mice that were left on the platform was shown. Further, the behavior of each mouse of the descending from the top of the platform to the floor was observed by classifying into the positive performing (=jumped down from the head) and the negative performing (=fall off from the tail).

FIG. 10(a) shows results of the observation of the state of each mouse on a cylindrical platform having a height of 20 cm and a diameter of 10 cm. The Gng7^{(+/Cre)} mouse that is a control mouse was not jumped down from the platform in 7 minutes. On the other hand, the HB-EGF deficient type TG mouse behaved in such ways of falling off from the tail in one minute after starting the test or of jumping down from the head.

FIG. 10(b) shows results of the ratio of the mice that were remained on the platform. In the mouse in which HB-EGF is not deficient, 80% or more of the mice were remained on the platform, on the other hand, in the HB-EGF deficient type mouse, only 20% of the mice were remained on the platform.

The backward fall observed in early time is considered to be due to the hyperactivity anxiety in which the situation where the mouse is put in cannot be recognized. Further, the front fall observed after one minute or more is a result of the impulsive behavior and is considered that the behavior cannot be restrained.

From the above results, it was understood that the HB-EGF deficient type mouse could not recognize the situation where the mouse is put in, showed the hyperactivity and anxiety, and took the impulsive behavior action.

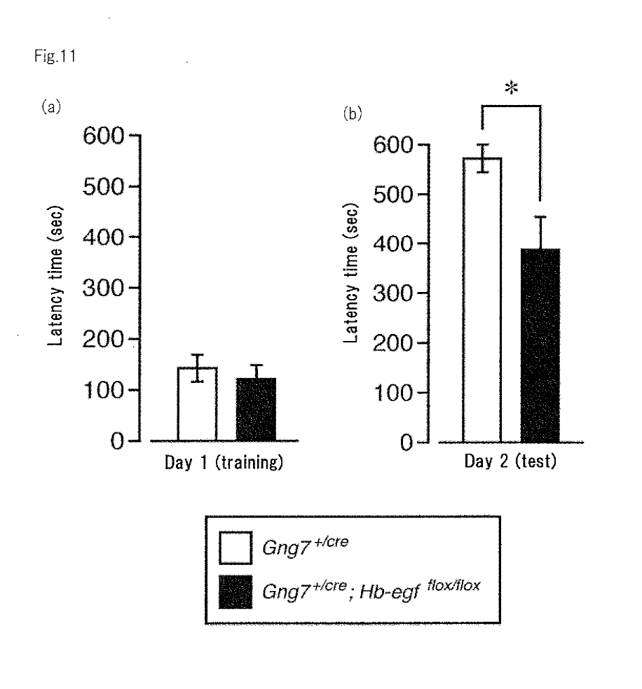

FIG. 11 shows results of the examination for each learned behavior of the HB-EGF deficient type TG mouse and the Gng7^{(+/+)}:Hb-egf^{(flox/flox)} mouse that is a control mouse.

In order to analyze the learned behavior of the mouse, a step-through test was performed. Each mouse was put in a light room of a step-through device, and the device was set up so that when the mouse moved through a movement hole to a dark room, the mouse receives an electric shock for two seconds from the electric grid carpeted on the floor. 24 hours after that, the mouse was again put in the light room, and the latency time until the mouse moved to the dark room was measured.

FIG. 11(a) shows results of the latency time on the first day of learning acquisition. Before conducting the learning acquisition, there was no difference between the times when each mouse moved from the light room to the dark room.

FIG. 11(b) shows results of the latency time on the second day of learning acquisition. It is understood that the latency time of the HB-EGF deficient type TG mouse was shorter than that of the Gng7^{(+/+)}):Hb-egf^{(flox/flox)} mouse that is a control mouse.

By learning that pain was associated when moving from the light room to the dark room, the Gng7^{(+/+)}:Hb-egf^{(flox/flox)} mouse that is a control mouse had the time when moving from the light room to the dark room longer, however, the HB-EGF deficient type TO mouse did not have the time longer than that of the Gng7^{(+/+)}:Hb-egf^{(flox/flox)} mouse that is a control mouse. From the above, it is understood that the HB-EGF deficient type TG mouse has a low learning acquisition ability.

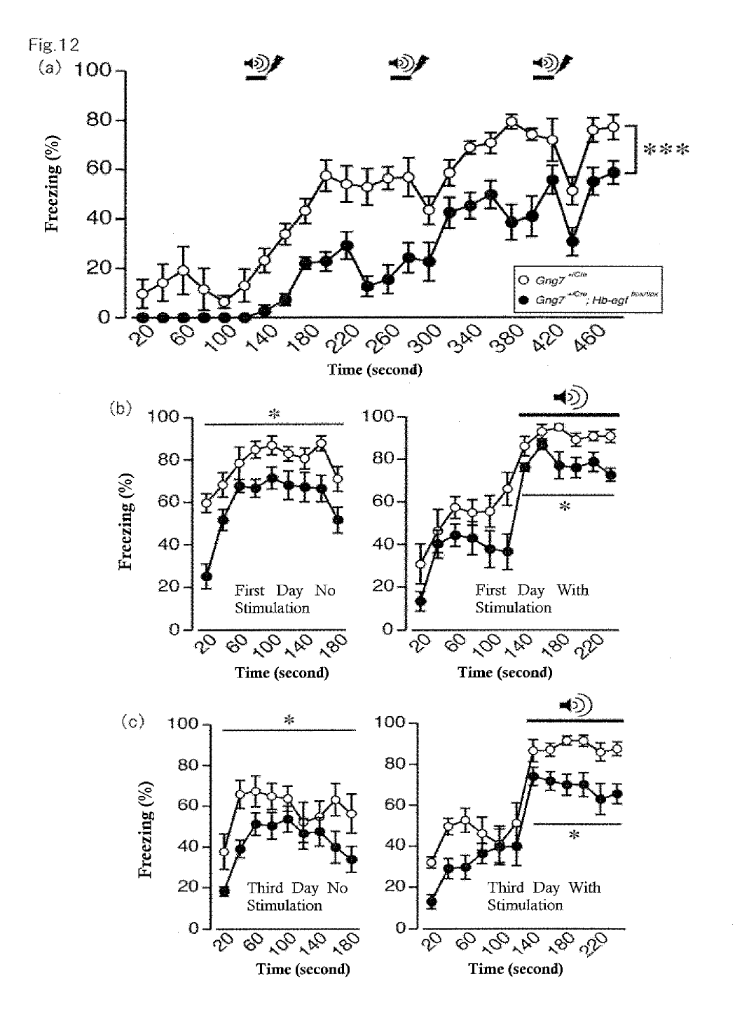

FIG. 12 shows results of the examination of each learned behavior of contextual fear conditioning of the HB-EGF deficient type TG mouse and the Gng7^{(+/+)}:Hb-egf^{(flox/flox)} mouse that is a control mouse.

In order to analyze the learned behavior of the mouse, a learning test of contextual fear conditioning was conducted. Each mouse was put in a square conditioning chamber in which the front was Plexiglas, both side and the back side were covered with grey walls and the floor was carpeted with electric grid, the mouse freely moved for 2 minutes, and then sounds at 4000 Hz and 80 dB were given for 20 seconds, and immediately after the sound the stimulation that gives electric shock at 0.5 mA for 2 seconds was added total tree times at an interval of 120 seconds. 24 hours, and 72 hours after that, the mouse was again put in the square conditioning chamber, and the freezing was measured for three minutes. After that, the mouse was put in a triangular test chamber, the whole surface of which was blue-and-white-striped walls, the mouse freely moved for 2 minutes, and then the freezing when sounds at 4000 Hz and 80 dB were given for 2 minutes was measured.

FIG. 12(a) shows results of the measurement of the freezing of each mouse to which electric shock was given total 3 times at an interval of 120 seconds. It is understood that the freezing time of the HB-EGF deficient type TG mouse was shorter than that of the Gng7^{(+/+)}:Hb-egf^{(flox/flox)} mouse that was a control mouse.

FIG. 12(b) shows results of the measurement of the freezing time of each mouse 24 hours and 72 hours after the total 3 times of the electric shock at an interval of 120 seconds. It is understood that in both cases of 24 hours and 72 hours after the electric shock, the freezing time of the HB-EGF deficient type TG mouse became short with or without sound stimulation.

From the above, it is suggested that the HB-EGF deficient type TG mouse caused not only simple learning disability but also the decrease of hyperactivity and broad-sense cognitive function.

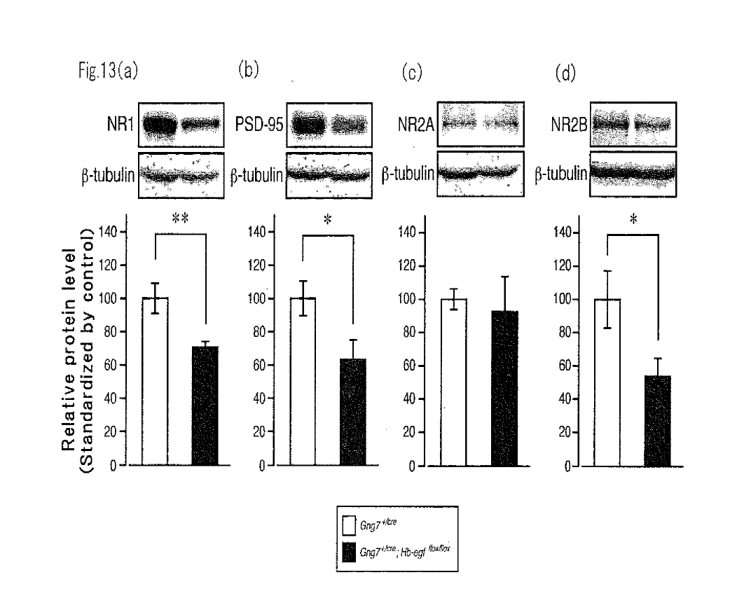

FIG. 13 shows results of the comparison of the expression level of the NMDA protein involved in brain function in the hippocampus. The expression level of the protein was determined by Western blot.

The expression level of the protein was determined in accordance with the following processes. First, each mouse was decapitated, and then the hippocampus region was expeditiously extracted on ice while avoiding the contaminants of the blood and hair. After that, SDS Sample buffer (50 mM, Tris-HCl (pH 6.8), 2% SDS, 10% Glycerol, 1 MAPMSF) was added, and ultrasonic breaking was performed on ice. After centrifuging (15000 rpm, 5 minutes, 4° C.), the supernatant was used as the total protein for analysis of NR1, NR2A, NR2B, PSD-95, β-tubulin. The protein determination was performed by using DcProtein Assay (Bio-Rad Laboratories, Hercules, Calif., USA). Using 15% polyacrylamide gel, 20 μg of sample was subjected to electrophoresis under the conditions of constant current (20 mA), and then the resultant was transcribed on a nitrocellulose membrane. The resultant was subjected to the blocking with 5% skim milk/2% FBS/TBS-T (20 mM Tris-HCl, pH 7.6, 150 mM NaCl, 0.1% Tween-20), and then reacted at 4° C. for 16 to 20 hours using various primary antibodies. Using a HRP (horseradish peroxidase) labeled antibody as the secondary antibody, the resultant was reacted at room temperature for 2 hours. For the detection, a chemiluminescence reagent for HRP (SuperSignal West Pico and Dura Chemiluminescent Substrate; Pierce Chemical, Rockford, Ill.) was used. As the endogenous control, PT-tubulin was used, and the resultant was quantified by calculating the relative expression level of the signal intensity obtained from the image analysis by ImageJ.

FIG. 13(a) shows results of the expression level of NR1 protein. FIG. 13(b) shows results of the expression level of PSD-95 protein. FIG. 13(c) shows results of the expression level of NR2A protein. FIG. 13(d) shows results of the expression level of NR2B protein.

As shown in FIGS. 13(a) to 13(d), it is understood that the decrease in each expression level of NR1, PSD-95 and NR2B was caused in the HB-EGF deficient type TG mouse. NR1, PSD-95 and NR2B are important proteins involved in learning and psychiatric disorder. Therefore, it was understood that the decrease of expression level of these proteins possibly caused neuropsychiatric disorder in the HB-EGF deficient type TG mouse.

ErbB4 is a cell receptor tyrosine kinase that is identified as a receptor of HB-EGF. The ErbB4 receptor controls the activation of a NMDA receptor via PSD-95 that is an tonotropic receptor anchoring protein. It is suggested that in the HB-EGF deficient type TG mouse in accordance with the present invention, the expression of an NMDA receptor via PSD-95 was decreased, and the abnormal behavior similar to obsessive-compulsive disorder, attention deficit hyperactivity disorder, anxiety-like behavior, and cognition disorder may be caused.

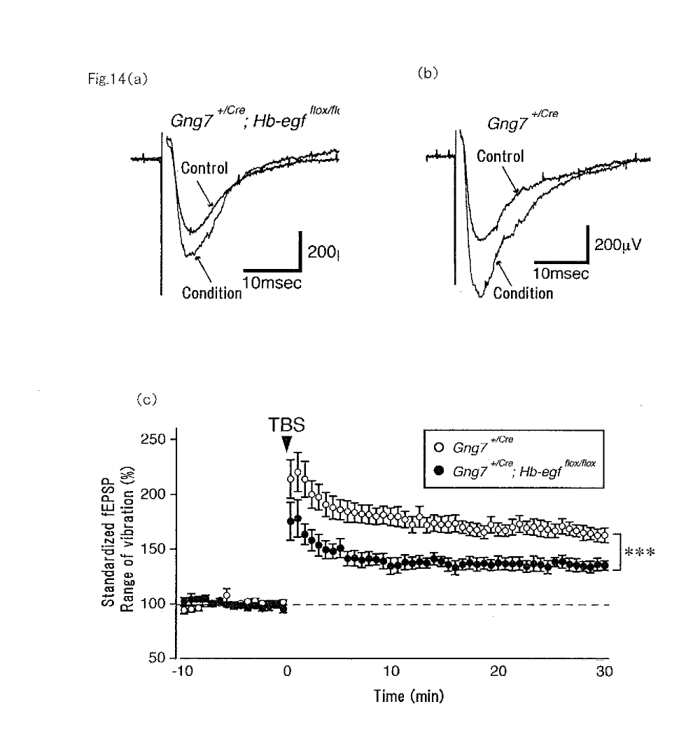

FIG. 14 shows results of the evaluation of synaptic plasticity in the hippocampal CA1 Region using LTP as an index. Synapse plays an important role in memory and learning, the decrease of the synaptic plasticity indicates a decrease in memory and learning ability. The synaptic plasticity can be evaluated by measuring the change of synaptic transmission rate due to the nervous excitement with electric stimulation.

In order to observe LTP, after acute decapitation, the hippocampus region of the brain in each mouse was sliced into thickness of 300 □m by a Vibratome, and the LTP phenomenon in the CA1 region was evaluated. After the observation (0 to 30 min) of electrical signals that are to be base line, theta burst stimulation (IBS) was performed, and the enhancement of the signals was evaluated up to a lapse of 30 minutes.

FIG. 14(a) shows results of the measurement of LTP for electric stimulation in the hippocampal CA1 Region in the HB-EGF deficient type TG mouse. FIG. 14(b) shows results of the measurement of LTP for electric stimulation in the hippocampal CA1 Region in the Gng7^{(+/+)}:Hb-egf^{(flox/flox)} mouse that is a control mouse. It is understood that the range of vibration of LTP under the conditions of FIG. 14(a) was smaller than the range of vibration of LTP under the conditions of FIG. 14(b). That is, it is understood that in the HB-EGF deficient type TG mouse, the synaptic plasticity in the hippocampal CA1 Region was decreased.

FIG. 14(c) shows results of the measurement of fEPSP slope by extracellular electrography. By stimulating to the hippocampal CA1 Region, neurotransmitter was released from presynaptic terminal. Extracellular electric field and electric potential that were changed by binding the released neurotransmitter to a postsynaptic receptor was recorded as field excitatory postsynaptic potential (fEPSP).

As shown in FIG. 14(c), it is understood that the amplification efficiency of fEPSP after the TBS was lower in the HB-EGF deficient type TG mouse. Therefore, it is suggested that in the HB-EGF deficient type TG mouse, the plasticity in the hippocampus region was also electrophysiologically decreased.

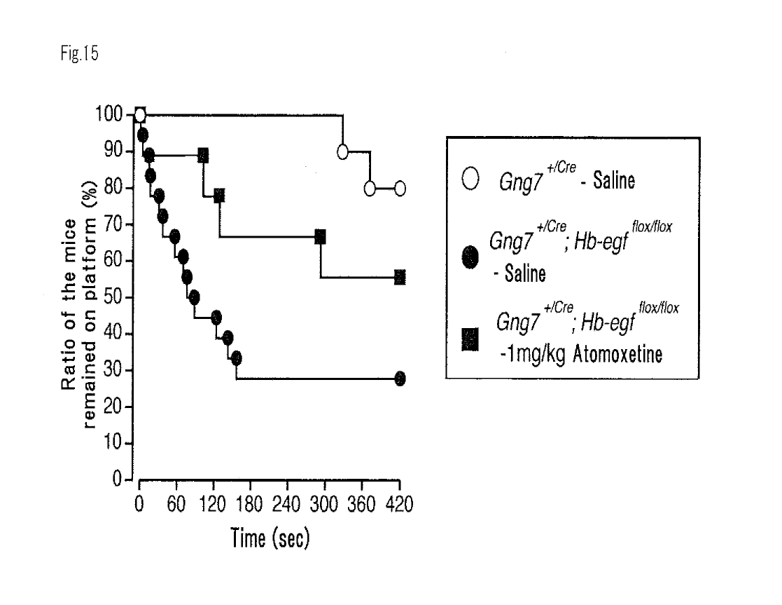

FIGS. 15 to 17 show results of the examination of the effects that are obtained by administering a therapeutic agent to each TG mouse in which neuropsychiatric disorder has been developed.

As the therapeutic agent, three drugs of atomoxetine (1 mg/kg), nefiracetam (1 mg/kg), and SA4503 (1 mg/kg) were used independently to administer into the abdominal cavity once a day for 7 days in a row. As a control of the therapeutic agent, saline was administered into the abdominal cavity once a day for 7 days in a row.

The atomoxetine is a noradrenaline reuptake inhibitor and is a drug increasing the noradrenaline and dopamine levels in the brain.

The nefiracetam is known as a brain function improving agent. The nefiracetam is a drug that has been developed as a pyrrolidone-based brain and neurotransmitter function improving agent that has a chemical structure similar to that of oxotremorine and lidocaine, and as a brain function improving agent that acts activating on the functional decrease of synapses of the acetylcholine (ACh) nervous system, the GABA nervous system, and the monoamine nervous system.

The SA4503 is a drug that binds to a receptor being present in a cell, a sigma receptor, and contributes to the functional recovery of the nerve cells that has been impaired.

FIG. 15 shows the ratio of the mice that were remained on the platform by observing the state of each mouse that had been arranged on the platform of a column having a height of 20 cm and a diameter of 10 cm. In the mouse in which HB-EGF is not deficient, 80% or more of the mice were remained on the platform. On the other hand, in the HB-EGF deficient type mouse, only 20% of the mice were remained on the platform. However, in the case of administering atomoxetine to the HB-EGF deficient type mouse, 50% or more of the mice were remained on the platform. Further, in the case of administering nefiracetam to the HB-EGF deficient type mouse, 70% or more of the mice were remained on the platform.

From the above results, it was understood that in the mouse to which nefiracetam or atomoxetine had been administered, the impulsivity was suppressed. By using HB-EGF deficient type TG mouse, a therapeutic agent that is effective for the suppression of impulsivity could be screened.

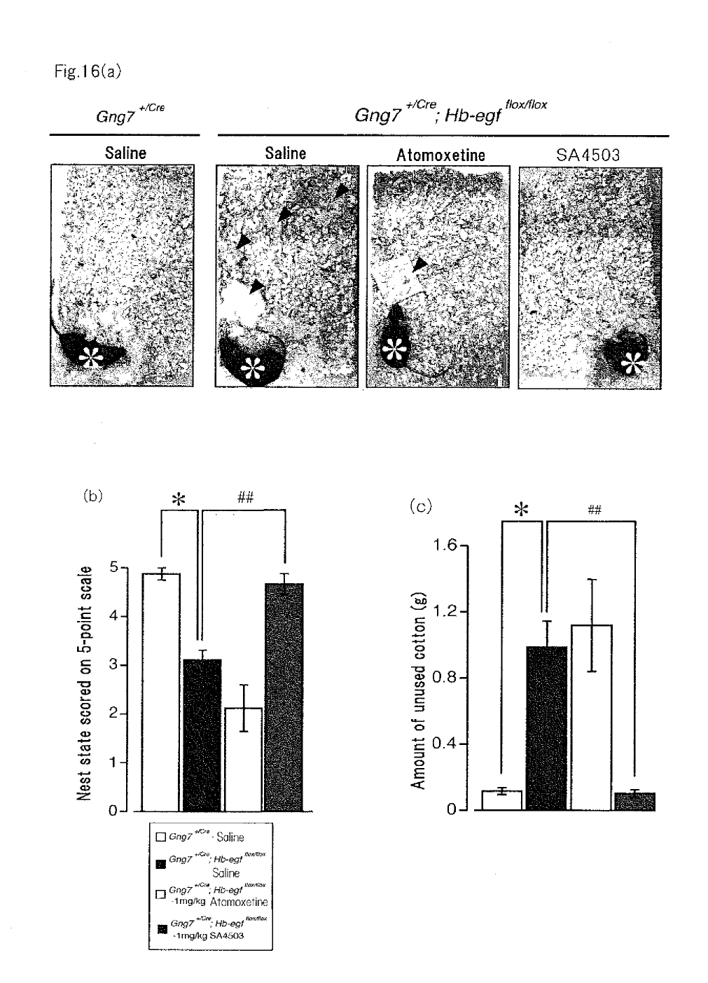

FIG. 16(a) shows the state of each nest that was made by the HB-EGF deficient type TG mouse and the mouse in which the HB-EGF is not deficient. The HB-EGF deficient type TG mouse to which saline or atomoxetine had been administered could not collect the cotton in one place and could not make the nest, on the other hand, the mouse to which nefiracetam or SA4503 had been administered could collect the cotton in one place and could make the nest.

FIG. 16(b) shows results of the evaluation on 5-point scale for the state of the nest that was made by each mouse. The rating scale was 3 or less for the nest that was made by the HB-EGF deficient type TG mouse to which saline or atomoxetine had been administered, on the other hand, the rating scale was 5 for the nest that was made by the HB-EGF deficient type TG mouse to which nefiracetam or SA4503 had been administered.

FIG. 16(c) shows the amount of the cotton that was not used for the nest-building in the cotton that is to be used by each mouse for the nest-building. The amount of the cotton that was not used was around 1.0 to 1.2 g for the HB-EGF deficient type TG mouse to which saline or atomoxetine had been administered, on the other hand, the amount of the cotton that was not used was around 0.1 g for the HB-EGF deficient type TG mouse to which nefiracetam or SA4503 had been administered, and was the same level as that of the mouse in which the HB-EGF is not deficient.

From the above results, it was understood that the mouse into which nefiracetam or SM 503 had been administered improved the social behavior, and the interest and consideration for the surrounding environment, and thus the treatment of the neuropsychiatric disorder was effectively performed. By using the HB-EGF deficient type TG mouse, a therapeutic agent that is effective for the improvement of social activity could be screened.

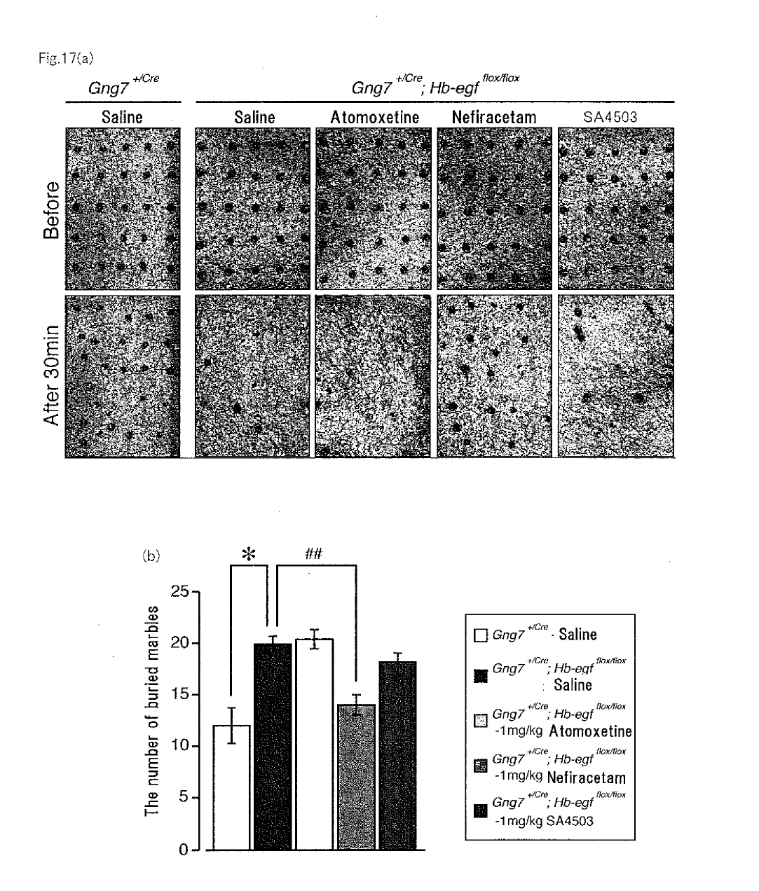

FIG. 17(a) shows the state of marbles before and after the marble-burying behavior test of the HB-EGF deficient type TG mouse and the mouse in which HB-EGF is not deficient. It is understood that each number of the marbles that were buried by the mouse in which HB-EGF is not deficient and by the mouse that is an HB-EGF deficient type TG mouse and into which nefiracetam had been administered was small.

FIG. 17(b) shows a graph of the number of the marbles that were buried. The number of the marbles that were buried by the HB-EGF deficient type TG mouse into which saline had been administered was large, on the other hand, the number of the marbles that were buried by the mouse into which nefiracetam had been administered was small. Further, the number of the marbles that were buried by the mouse into which atomoxetine or SA4503 had been administered, was the same level as that of the HB-EGF deficient type TG mouse into which saline had been administered.

From the above results, it was understood that in the mouse in which nefiracetam had been administered, the anxiety-like behavior related to obsessive-compulsive disorder was decreased. By using an HB-EGF deficient type TG mouse, a therapeutic agent that is effective for obsessive-compulsive disorder could be screened.

### INDUSTRIAL APPLICABILITY

The present invention relates to a therapeutic agent for neuropsychiatric disorder, therefore, the present invention can be used in pharmaceutical industry. Further, the present invention can be used for the research and development business of drug discovery for the treatment of neuropsychiatric disorder.

### SEQUENCE LISTING FREE TEXT

SEQ ID NO: 1 is an amino acid sequence of a Cre protein.

SEQ ID NO: 2 is a LoxP sequence.

SEQ ID NO: 3 is a sequence of primer.

SEQ ID NO: 4 is a sequence of primer.

SEQ ID NO: 5 is a sequence of primer.

SEQ ID NO: 6 is a sequence of primer.

SEQ ID NO: 7 is a sequence of primer.

SEQ ID NO: 8 is a sequence of primer.

SEQ ID NO: 9 is a sequence of primer.

### SEQUENCE LISTING

Sequence_ST25.txt

1. A transgenic animal other than human,
wherein an HB-EGF gene is deficient in hippocampal neuron region thereof, thereby the deficient causes neuropsychiatric disorder.

2. The transgenic animal other than human in accordance with claim 1,
wherein the transgenic aminal is obtained by crossbreeding a first transgenic animal other than human that contains a gene promoter being specifically expressed in a hippocampal neuron region and a Cre gene sequence, and a second transgenic animal other than human that contains an HB-EGF gene sequence sandwiched in between LoxP sequences.

3. The transgenic animal other than human in accordance with claim 2,
wherein the gene promoter is a Gng7 promoter, a CamK II promoter, or a Emx1 promoter.

4. The transgenic animal other than human in accordance with claim 1,
wherein the neuropsychiatric disorder is any one of depression, Alzheimer disease, psychiatric disorder, learning disability, and long-term memory impairment.

5. The transgenic animal other than human in accordance with claim 1,
wherein the transgenic animal other than human is a mouse.

6. A method for screening a therapeutic agent for neuropsychiatric disorder,
administrating a test substance to the transgenic animal other than human in accordance with claim 1 in which an HB-EGF gene is deficient, and
examining an effect of improvement of condition of neuropsychiatric disorder.

7. A method for producing a transgenic animal other than human,
crossbreeding an HB-EGFa first transgenic animal other than human and a second transgenic animal other than human,
wherein the first transgenic animal contains a gene promoter being specifically expressed in a hippocampal neuron region, the first transgenic animal containing a Cre gene sequence, and
the second transgenic animal contains an HB-EGF gene sandwiched in between LoxP sequences,
thereby a transgenic animal wherein an HB-EGF gene is deficient is obtained.

8. The transgenic animal other than human in accordance with claim 2,
wherein the transgenic animal other than human is a mouse.

9. The transgenic animal other than human in accordance with claim 3,
wherein the transgenic animal other than human is a mouse.

10. The transgenic animal other than human in accordance with claim 4,
wherein the transgenic animal other than human is a mouse.

11. A method for screening a therapeutic agent for neuropsychiatric disorder,
administrating a test substance to the transgenic animal other than human in accordance with claim 2 in which an HB-EGF gene is deficient, and
examining an effect of improvement of condition of neuropsychiatric disorder.

12. A method for screening a therapeutic agent for neuropsychiatric disorder,
administrating a test substance to the transgenic animal other than human in accordance with claim 3 in which an HB-EGF gene is deficient, and
examining an effect of improvement of condition of neuropsychiatric disorder.

14. A method for screening a therapeutic agent for neuropsychiatric disorder,
administrating a test substance to the transgenic animal other than human in accordance with claim 4 in which an HB-EGF gene is deficient, and
examining an effect of improvement of condition of neuropsychiatric disorder.

15. A method for screening a therapeutic agent for neuropsychiatric disorder,
administrating a test substance to the transgenic animal other than human in accordance with claim 5 in which an HB-EGF gene is deficient, and
examining an effect of improvement of condition of neuropsychiatric disorder.


SEQUENCE LISTING (9 sequences)

SEQ ID NO: 1 - protein, 345 aa, Bacteriophage P1
MVQTSLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - DNA, 34 bp, Mouse
ataacttcgtatagcatacattatacgaagttat

SEQ ID NO: 3 - DNA, 22 bp, Artificial Sequence
  Primer
ggcgacgttgttagtacctgac

SEQ ID NO: 4 - DNA, 25 bp, Artificial Sequence
  Primer
atccctgaacatgtccatcaggttc

SEQ ID NO: 5 - DNA, 30 bp, Artificial Sequence
  Primer
tataggtacccagaagtgaattcggttcgc

SEQ ID NO: 6 - DNA, 20 bp, Artificial Sequence
  Primer
atgggatcggccattgaaca

SEQ ID NO: 7 - DNA, 20 bp, Artificial Sequence
  Primer
gaagaactcgtcaagaaggc

SEQ ID NO: 8 - DNA, 30 bp, Artificial Sequence
  Primer
agggcaagatcatgtgtcctgcctccagcc

SEQ ID NO: 9 - DNA, 31 bp, Artificial Sequence
  Primer
catgatgctccagtgagtaggctctgattac